(19) United States
(12) Patent Application Publication
(10) Pub. No.: US 2023/0323305 A1
(43) Pub. Date: Oct. 12, 2023

(54) BIOINK CONTAINING SELF-ASSEMBLING PEPTIDES

(71) Applicants: 3-D Matrix, Ltd., Cambridge, MA (US); CENTRE NATIONAL DE LA RECHERCHE SCIENTIFIQUE (CNRS), PARIS CEDEX 16 (FR); UNIVERSITE CLAUDE BERNARD LYON 1 (UCBL1), VILLEURBANNE (FR); L’ECOLE SUPERIEURE DE CHIMIE, PHYSIQUE, ELECTRONIQUE DE LYON (CPE Lyon), Villeurbanne Cedex (FR)

(72) Inventors: Christophe A. Marquette, Villeurbanne (FR); Marie Reveiller Buffier, Fontaines Saint Martin (FR); Marika G. Rioult, Cambridge, MA (US); Eun Seok Gil, Acton, MA (US)

(73) Assignees: 3-D Matrix, Ltd., Tokyo (JP); CENTRE NATIONAL DE LA RECHERCHE SCIENTIFIQUE (CNRS), 75794 PARIS CEDEX 16 (FR); UNIVERSITE CLAUDE BERNARD LYON 1 (UCBL1), VILLEURBANNE (FR); L’ECOLE SUPERIEURE DE CHIMIE, PHYSIQUE, ELECTRONIQUE DE LYON (CPE Lyon), Villeurbanne Cedex (FR)

(21) Appl. No.: 18/296,158

(22) Filed: Apr. 5, 2023

Related U.S. Application Data
(60) Provisional application No. 63/327,825, filed on Apr. 6, 2022.

(51) Int. Cl. C12N 5/077; B33Y 70/00

(52) U.S. Cl. CPC C12N 5/0656; B33Y 70/00; C12N 2513/00

(57) ABSTRACT

A bioink comprises a cell solution comprising living cells and a cell culture medium, and a self-assembling peptide solution comprising a self-assembling peptide. The bioink is formed by mixing and extruding the cell solution and the self-assembling peptide solution together, and the bioink can be continuously extruded from the mixer after mixing. Useful methods of making bioinks and are also disclosed.

### PRIORITY

This application claims priority to U.S. provisional Application No. 63/327,825, filed Apr. 6, 2022, the entire contents of which are hereby incorporated by reference.

### SEQUENCE LISTING

The instant application contains an XML Sequence Listing which has been submitted electronically and is hereby incorporated by reference in its entirety. The Sequence Listing, created on Mar. 20, 2023, is named 3DM-21-02-3DP1-PCT-SL.xml and is 30,429 bytes in size.

### FIELD OF THE INVENTION

This invention relates to bioinks suitable for 3D printing, and more particularly to improved bioinks containing self-assembling peptides.

### BACKGROUND OF THE INVENTION

3-D printing of tissues involves the assembly of cells into a controlled volume, where the bioink mimics in vivo characteristics of tissues. There are competing design constraints which such bioinks. For example, a particular shape is desired, and so the material used to make that shape must be extrudable but also ultimately capable of holding that shape. But adding the requirement of living cells means the material used and the way the material is extruded must also be non-toxic to the living cells. Various efforts are known to make bioinks. These include seeding cells on printed structures, mixing cells with a printing medium and then printed into desired structures, and printing cell clusters (spheroids) and stringing them together to form functional tissues. As this technology is fairly new, different printers and printing techniques have been developed to print the cells and the matrix. The commonly used methods of bioprinting are extrusion, laser, inkjet, and tissue fragment printing, all with a goal of positioning of the living cells and/or the biomaterials, and to create functional tissue analogs.

Many different materials have been used as bioinks, including, for example, natural materials such as alginate, gelatin, collagen, silk, gellan gum, hyaluronic acid, dextran, and cellulose; synthetic materials like polycaprolactone, pluronic acid and polyethylene glycol; and commercial materials like Derma Matrix®, Novogel®, and CELLINK®. Bioinks should be printable, with easy and cell friendly cross-linking after printing/extrusion so as to retain stiffness quickly after extrusion, should hold its shape so as to mimic the desired shape with precision, should be biocompatible to not just allow cells to live but also allow for cell growth and not cause an immune response, and should biodegradable. Further, such bioinks should preferably mimic the native tissue environment for cells to attach and/or grow within, and such bioinks should be customizable so as to be modifiable for different applications (such as soft, medium or hard tissues). In spite of the numerous efforts in the advancement of the bioprinting technology, the development of a satisfactory bioink which meets all the requirements to create a biomaterial serving as a suitable functional tissue analog has been limited. It would be desirable to provide a bioink which goes further towards satisfying these competing design constraints.

### SUMMARY OF THE INVENTION

In accordance with a first aspect, a bioink comprises a cell solution comprising living cells and a cell culture medium, and a self-assembling peptide solution comprising a self-assembling peptide. The bioink is formed by mixing and extruding the cell solution and the self-assembling peptide solution together, and the bioink is extruded from the mixer after mixing. Useful methods of making bioinks are also disclosed and described in further detail below.

From the foregoing disclosure and the following more detailed description of various embodiments it will be apparent to those skilled in the art that the present invention provides a significant advance in the technology of bioinks. Particularly significant in this regard is the potential the invention affords for providing a high quality bioink which is easily extrudable, customizable and is non-toxic to living cells. Additional features and advantages of various embodiments will be better understood in view of the detailed description provided below.

### DETAILED DESCRIPTION OF CERTAIN EMBODIMENTS

It will be apparent to those skilled in the art, that is, to those who have knowledge or experience in this area of technology, that many uses and design variations are possible for the bioink disclosed here. The following detailed discussion of various alternate features and embodiments will illustrate the general principles of the invention with reference to 3-D printing of bioinks, and more particularly to medical and research applications of such bioinks. Other embodiments suitable for other applications will be apparent to those skilled in the art given the benefit of this disclosure.

In accordance with a first aspect, there is provided a bioink formed from a combination of a self-assembling peptide solution and a cell solution. The self-assembling peptide solution comprises one of many self-assembling peptide, for example, RADA16, which a peptide of 16 amino acids in length with protective groups on both ends (AcN-RADARADARADARADA-CNH₂). An example of suitable, commercially available, self-assembling peptide solution is PuraStat®, from 3-D Matrix Medical Technology, Inc., which is an aqueous solution of about 2.5% RADA16. RADA16 has alternating hydrophobic (alanine) and hydrophilic (arginine and aspartic acid) groups which allow for organization into stable β-sheets. The resulting porous hydrogels contain high water content (on the order of 99%) and resemble the extracellular matrix of native body tissue. Optionally a sucrose solution may be incorporated into the self-assembling peptide solution. Other self-assembling peptides, including shorter RADA derivatives (RADAR, RADA12, for example), peptidomimetics (such as D-forms of the amino acids, for example), peptide amphiphiles, other concentrations, and other self-assembling peptide solutions suitable for use in a bioink will be readily apparent to those skilled in the art given the benefit of this disclosure.

In some embodiments, the SAPs comprise a sequence of amino acid residues conforming to one or more of Formulas I-IV:

((Xaaneu-Xaa+)x(Xaaneu-Xaa−)y)n (I)

((Xaaneu-Xaa−)x(Xaaneu-Xaa+)y)n (II)

((Xaa+-Xaaneu)x(Xaa−-Xaaneu)y)n (III)

((Xaa−-Xaaneu)x(Xaa+-Xaaneu)y)n (IV)

Xaaneu represents an amino acid residue having a neutral charge; Xaa+ represents an amino acid residue having a positive charge; Xaa− represents an amino acid residue having a negative charge; x and y are integers having a value of 1, 2, 3, or 4, independently; and n is an integer having a value of 1-5.

In some embodiments, the SAPs further comprise an amino acid sequence that interacts with the extracellular matrix, wherein the amino acid sequence anchors the SAPs to the extracellular matrix.

In some embodiments, the amino acid residues in the SAPs can be naturally occurring or non-naturally occurring amino acid residues. Naturally occurring amino acids can include amino acid residues encoded by the standard genetic code as well as non-standard amino acids (e.g., amino acids having the D-configuration instead of the L-configuration), as well as those amino acids that can be formed by modifications of standard amino acids (e.g., pyrolysine or selenocysteine). Suitable non-naturally occurring amino acids include, but are not limited to, D-alloisoleucine(2R,3S)-2-amino-3-methylpentanoic acid, L-cyclopentyl glycine (S)-2-amino-2-cyclopentyl acetic acid.

In some embodiments, the specific peptides for use in the method of the present invention can be chosen from one or more peptides listed in the Table 1 below.

TABLE 1

| Self-assembling Peptide | SEQ ID NO: |
|---|---|
| RADARADARADARADA | SEQ ID NO: 1 |
| KLDKLDKLDKLD | SEQ ID NO: 2 |
| IEIKIEIKIEIKI | SEQ ID NO: 3 |
| QLELQLELQLEL | SEQ ID NO: 4 |
| ADARADARADARADAR | SEQ ID NO: 5 |
| RAEARAEARAEARAEA | SEQ ID NO: 6 |
| RVDVRVDVRVDVRVDV | SEQ ID NO: 7 |
| RLDLRLDLRLDLRLDL | SEQ ID NO: 8 |
| RIDIRIDIRIDIRIDI | SEQ ID NO: 9 |
| RFDFRFDFRFDFRFDF | SEQ ID NO: 10 |
| AEARAEARAEARAEAR | SEQ ID NO: 11 |
| KADAKADAKADAKADA | SEQ ID NO: 12 |
| KIDIKIDIKIDIKIDI | SEQ ID NO: 13 |
| KIEIKIEIKIEIKIEI | SEQ ID NO: 14 |
| IDIKIDIKIDIKI | SEQ ID NO: 15 |
| IEIRIEIRIEIRI | SEQ ID NO: 16 |
| LELKLELKLELKL | SEQ ID NO: 17 |
| FEFKFEFKFEFKF | SEQ ID NO: 18 |
| KLDLKLDLKLDL | SEQ ID NO: 19 |
| KLELKLELKLEL | SEQ ID NO: 20 |
| FEFRFEFRFEFRF | SEQ ID NO: 21 |
| YEYKYEYKYEYKY | SEQ ID NO: 22 |
| WEWKWEWKWEWKW | SEQ ID NO: 23 |

In other embodiments, another class of materials that can self-assemble are peptidomimetics. Peptidomimetics, as used herein, refers to molecules which mimic peptide structure. Peptidomimetics have general features analogous to their parent structures, polypeptides, such as amphiphilicity. Examples of such peptidomimetic materials are described in Moore et al., Chem. Rev. 101(12), 3893-4012 (2001). The peptidomimetic materials can be classified into four categories: α-peptides, β-peptides, γ-peptides, and δ-peptides. Copolymers of these peptides can also be used. Examples of α-peptide peptidomimetics include, but are not limited to, N,N′-linked oligoureas, oligopyrrolinones, oxazolidin-2-ones, azatides and azapeptides. Examples of β-peptides include, but are not limited to, β-peptide foldamers, α-aminoxy acids, sulfur-containing β-peptide analogues, and hydrazino peptides. Examples of γ-peptides include, but are not limited to, γ-peptide foldamers, oligoureas, oligocarbamates, and phosphodiesters. Examples of δ-peptides include, but are not limited to, alkene-based δ-amino acids and carbopeptoids, such as pyranose-based carbopeptoids and furanose-based carbopeptoids.

In certain embodiments, the SAP is AC5®, AC5-V® or AC5-G™ made by Arch Therapeutics, Inc. (see www.archtherapeutics.com).

The cell solution may comprise any one of several living cells or combinations of living cells. Examples are numerous and comprise stomal vascular fraction (SVF) cells, stem cells, tumor cells, hepatocyte progenitor cells, rat pheochromocytoma cells (PC12), hippocampal neurons, endothelial cells, neuronal cells, fibroblasts, keratinocytes, and transformed cells including, for example, MG-62, SH-SYSY, HEK293, NIH3T3). Other cells suitable for use in the cell solution of the bioinks disclosed herein will be readily apparent to those skilled in the art given the benefit of this disclosure.

The cell solution would typically contain a cell culture medium to provide nutrition to the cells and maintain a healthy environment. Suitable components of a cell culture medium can comprise, for example, Dulbecco's Modified Eagle Medium (DMEM), which is a widely used basal medium for supporting the growth of many different mammalian cells, fetal bovine serum, Minimal Essential Medium (MEM) nonessential amino acids, L-glutamine solution, antibiotics such as penicillin and streptomycin, and a trypsin-EDTA (Ethylenediaminetetraacetic acid) solution. Other cell culture mediums components suitable for use in the cell solution of the bioinks disclosed herein will be readily apparent to those skilled in the art given the benefit of this disclosure.

In accordance with one embodiment, the bioink can be formed by combining the self-assembling peptide solution together with the cell solution in a mixer. After mixing together to form the bioink, the bioink may be extruded. Preferably this occurs immediately or continuously. Continuously is understood here to mean that the addition of the self-assembling peptide solution and/or the cell solution to the mixer forces or urges a mixed material bioink to be extruded from the mixer. This of course assumes that a control valve on a nozzle of the extruder is open to allow the mixture to flow from the chamber and out the nozzle. Typically, the time involved between mixing and extrusion is relatively short, such as no more than a minute after mixing, and typically only a few seconds. That is, typically the mixer/extruder may have a chamber which receives both the self-assembling peptide solution and the cell solution, and where mixing occurs, but the mixture will only be present in the chamber briefly before moving to the nozzle. Generally, as the pH of the self-assembling peptide solution rises, as by mixing with a cell solution to form a bioink, cross linking occurs. It is preferable to have the bioink extruded quickly such that most of the cross linking occurs after the bioink has been extruded and formed a shaped object. Optionally a consolidation solution, such as one containing NaOH, may be added to help control the pH of the bioink. A ratio of 0.5-1.5/100 (consolidation solution/self-assembling peptide solution, such as NaOH 1N/RADA16) can be used in the extruder, more preferably 1/100 (NaOH 1N/RADA16), and preferably mixed together. The consolidation solution can be loaded, under sterile conditions, in a 10 mL syringe, for example, just before being connected to the mixing extruder. In normal operation the self-assembling peptide solution and the cell solution (and any consolidation solution) are continuously introduced together and mixed, and the act of introducing more of the cell solution and the self-assembling peptide solution together forces mixed bioink to be extruded through a nozzle into an object having a desired shape. Suitable examples of a mixer can comprise, for example, the T333 classical FDM (“fused deposition modeling”) 3-D printer made by the French corporation Tobeca, provided with a mixing chamber to receive both solutions, and an adjacent 1 mm nozzle such that introduction of more of either the self-assembling peptide solution, the cell solution, or both immediately forces the bioink through the nozzle. The volume of mixture can be controlled, along with the location, and can be controlled concurrently. Other mixers and 3-D printers suitable for mixing and extruding bioinks will be readily apparent to those skilled in the art given the benefit of this disclosure. Optionally the bioink may be at least partially immersed in an immersion solution after extrusion to provide additional nutrients to help keep the cells in the bioink alive.

Typically a ratio of the self-assembling peptide solution to the cell solution which are mixed together to form the bioink is between 6:1 and 20:1; 8 to 1 and 13:1, or about 10:1 by volume. Advantageously, the bioink preferably can be free of any additional thickening agent, as the cross-linking of the self-assembling peptide solution creates an object with sufficient rigidity/viscosity for many applications. Further, the self-assembling peptide solution may be kept chilled or may be at room temperature (that is, at least above 5° C., above 10° C., or above 15° C.) prior to mixing with the cell solution, and/or after mixing with the cell solution, but before the step of extruding the bioink. The cell solution can be kept at the temperature the cells normally live in, which for cells that live in a human is around 35-40° C., and more specifically near 37° C., for example. The bioink can be a mixture of the self-assembling peptide solution and the cell solution which cures to form an object having a desired or pre-programmed shape. The object cures spontaneously after extrusion, without additional heating or other process steps by an operator.

Preferably during both the steps of either mixing, extruding, or both mixing and extruding, a shear stress on the bioink is kept below a viability limit for the living cells, such as, for example, 4000 PA. The shear stress may be controlled by restricting the rate of mixing and/or extruding. The bioink may also be immersed in an immersion solution after extrusion to help and/or to protect the cells present. The immersion solution can comprise, for example, a basal medium for supporting the growth of many different types of cells, especially mammalian cells, such as Dulbecco's Modified Eagle Medium (DMEM). Other compositions and combinations of chemicals suitable for use as the immersion solution will be readily apparent to those skilled in the art given the benefit of this disclosure.

EXAMPLE 1. One example of a mixed bioink is as follows. A 2.5% (w/v) RADA16 self-assembling peptide solution was loaded into a mixing extruder at a ratio of about 10:1 RADA16 to cell solution by volume. RADA16 was loaded, under sterile and ambient conditions, just before being connected to the mixing extruder. The cell solution comprises green fluorescent protein (GFP)-expressing fibroblasts (NIH3T3/GFP, AKR-214, Cell Biolabs Inc., US), which were expanded and suspended in DMEM (high glucose) from Gibco (France) and supplemented with 10% (v/v) fetal bovine serum (FBS) from Gibco (France), 0.1 mM MEM non-essential amino acids (NEAA) from Invitrogen (France), 2 mM L-glutamine from Gibco (France) and 1% (w/v) penicillin/streptomycin (10,000 U/mL) from Gibco (France). Before introduction to the mixer/extruder, the cells the solution were trypsinized (0.25% (v/v) trypsin-EDTA from ThermoFisher (France)), and counted. A cell solution having about 2.3×10⁷ cells/mL was connected to the mixing extruder. After introducing the self-assembling peptide solution together with the cell solution in the mixer, the resulting bioink was extruded into a shaped object. After bioprinting, the shaped object was immersed in an immersion solution of 5 mL of DMEM (high glucose) from Gibco (France) supplemented with 10% (v/v) fetal bovine serum (FBS) from Gibco (France), 0.1 mM MEM non-essential amino acids (NEAA) from Invitrogen, France), 2 mM L-glutamine from Gibco (France) and 1% (w/v) penicillin/streptomycin (10,000 U/mL) from Gibco (France), and placed at 37° C. in a 5% CO² incubator to keep the cells warm at this preferred temperature. The NIH 3T3 eGFP mouse fibroblasts were maintained in this state and were observed to be viable even after 60 days. Maximal shear stress was measured to be 3063 PA. Another important observation is that the pH of the self-assembling peptide solution is quickly neutralized by the ions in the living cell solution during the co-extrusion process. This indicated that the cells were not affected by the initial acidic pH of the peptide. The structural integrity of the shaped objects formed by the bioinks was maintained.

From the foregoing disclosure and detailed description of certain embodiments, it will be apparent that various modifications, additions and other alternative embodiments are possible without departing from the true scope of the invention. The embodiments discussed were chosen and described to provide the best illustration of the principles of the invention and its practical application to thereby enable one of ordinary skill in the art to use the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A bioink comprising, in combination:
a cell solution comprising living cells and a cell culture medium; and
a self-assembling peptide solution comprising a self-assembling peptide;
wherein the bioink is formed by mixing and extruding the cell solution and the self-assembling peptide solution together, and the bioink is extruded from the mixer after mixing.

2. The bioink of claim 1 wherein a ratio of the self-assembling peptide solution to the cell solution is between 6 to 1 and 20 to 1 by volume.

3. The bioink of claim 2 wherein the ratio is between 8 to 1 and 13 to 1 by volume.

4. The bioink of claim 1 wherein the self-assembling peptide is chosen from Table 1.

5. The bioink of claim 1 wherein the bioink is free of a thickening agent.

6. The bioink of claim 1 wherein introduction of one of the self-assembling peptide solution, the cell solution, or both to the mixer urges the bioink to be extruded from the mixer.

7. The bioink of claim 1 further comprising an immersion solution, wherein the bioink is at least partially immersed in the immersion solution.

8. The bioink of claim 1 further comprising a consolidation solution to adjust pH added to the combination of the cell solution and the self-assembling peptide solution prior to mixing and extruding.

9. A method of making a bioink comprising, in combination:
mixing a cell solution comprising living cells and a cell culture medium with a self-assembling peptide solution comprising a self-assembling peptide in a mixer to form a mixture;
extruding the mixture to form an object; and
curing the object.

10. The method of making the bioink of claim 9 wherein the mixture is continuously extruded from the mixer after mixing.

11. The method of making the bioink of claim 9 wherein the step of curing occurs spontaneously after mixing the cell solution with the self-assembling peptide solution.

12. The method of claim 9 wherein an extruder defines a chamber adjacent a nozzle, and the step of mixing occurs in the chamber.

13. The method of claim 9 wherein the step of extruding comprises extruding the bioink through the nozzle.

14. The method of claim 9 wherein during the steps of mixing and extruding, a shear stress on the bioink is kept below a viability limit for the living cells.

15. The method of claim 9 further comprising immersing the object in an immersion solution.

16. The method of claim 4, wherein the self-assembling peptide is RADA16 (SEQ ID NO:1).


SEQUENCE LISTING (23 sequences)

SEQ ID NO: 1 - protein, 16 aa, Synthetic construct
  REGION 1..16: note = Description of Artificial Sequence: Synthetic Peptide
RADARADARADARADA

SEQ ID NO: 2 - protein, 12 aa, Synthetic construct
  REGION 1..12: note = Description of Artificial Sequence: Synthetic Peptide
KLDKLDKLDKLD

SEQ ID NO: 3 - protein, 13 aa, Synthetic construct
  REGION 1..13: note = Description of Artificial Sequence: Synthetic Peptide
IEIKIEIKIEIKI

SEQ ID NO: 4 - protein, 12 aa, Synthetic construct
  REGION 1..12: note = Description of Artificial Sequence: Synthetic Peptide
QLELQLELQLEL

SEQ ID NO: 5 - protein, 16 aa, Synthetic construct
  REGION 1..16: note = Description of Artificial Sequence: Synthetic Peptide
ADARADARADARADAR

SEQ ID NO: 6 - protein, 16 aa, Synthetic construct
  REGION 1..16: note = Description of Artificial Sequence: Synthetic Peptide
RAEARAEARAEARAEA

SEQ ID NO: 7 - protein, 16 aa, Synthetic construct
  REGION 1..16: note = Description of Artificial Sequence: Synthetic Peptide
RVDVRVDVRVDVRVDV

SEQ ID NO: 8 - protein, 16 aa, Synthetic construct
  REGION 1..16: note = Description of Artificial Sequence: Synthetic Peptide
RLDLRLDLRLDLRLDL

SEQ ID NO: 9 - protein, 16 aa, Synthetic construct
  REGION 1..16: note = Description of Artificial Sequence: Synthetic Peptide
RIDIRIDIRIDIRIDI

SEQ ID NO: 10 - protein, 16 aa, Synthetic construct
  REGION 1..16: note = Description of Artificial Sequence: Synthetic Peptide
RFDFRFDFRFDFRFDF

SEQ ID NO: 11 - protein, 16 aa, Synthetic construct
  REGION 1..16: note = Description of Artificial Sequence: Synthetic Peptide
AEARAEARAEARAEAR

SEQ ID NO: 12 - protein, 16 aa, Synthetic construct
  REGION 1..16: note = Description of Artificial Sequence: Synthetic Peptide
KADAKADAKADAKADA

SEQ ID NO: 13 - protein, 16 aa, Synthetic construct
  REGION 1..16: note = Description of Artificial Sequence: Synthetic Peptide
KIDIKIDIKIDIKIDI

SEQ ID NO: 14 - protein, 16 aa, Synthetic construct
  REGION 1..16: note = Description of Artificial Sequence: Synthetic Peptide
KIEIKIEIKIEIKIEI

SEQ ID NO: 15 - protein, 13 aa, Synthetic construct
  REGION 1..13: note = Description of Artificial Sequence: Synthetic Peptide
IDIKIDIKIDIKI

SEQ ID NO: 16 - protein, 13 aa, Synthetic construct
  REGION 1..13: note = Description of Artificial Sequence: Synthetic Peptide
IEIRIEIRIEIRI

SEQ ID NO: 17 - protein, 13 aa, Synthetic construct
  REGION 1..13: note = Description of Artificial Sequence: Synthetic Peptide
LELKLELKLELKL

SEQ ID NO: 18 - protein, 13 aa, Synthetic construct
  REGION 1..13: note = Description of Artificial Sequence: Synthetic Peptide
FEFKFEFKFEFKF

SEQ ID NO: 19 - protein, 12 aa, Synthetic construct
  REGION 1..12: note = Description of Artificial Sequence: Synthetic Peptide
KLDLKLDLKLDL

SEQ ID NO: 20 - protein, 12 aa, Synthetic construct
  REGION 1..12: note = Description of Artificial Sequence: Synthetic Peptide
KLELKLELKLEL

SEQ ID NO: 21 - protein, 13 aa, Synthetic construct
  REGION 1..13: note = Description of Artificial Sequence: Synthetic Peptide
FEFRFEFRFEFRF

SEQ ID NO: 22 - protein, 13 aa, Synthetic construct
  REGION 1..13: note = Description of Artificial Sequence: Synthetic Peptide
YEYKYEYKYEYKY

SEQ ID NO: 23 - protein, 13 aa, Synthetic construct
  REGION 1..13: note = Description of Artificial Sequence: Synthetic Peptide
WEWKWEWKWEWKW